(19) United States
(12) Patent Application Publication
(10) Pub. No.: US 2023/0149664 A1
(43) Pub. Date: May 18, 2023

(54) SHEATH/CATHETER SECURING DEVICE

(71) Applicant: UNIVERSITY OF FLORIDA RESEARCH FOUNDATION, INCORPORATED, Gainesville, FL (US)

(72) Inventor: Travis Eugene MEYER, Jacksonville, FL (US)

(21) Appl. No.: 17/918,052

(22) PCT Filed: Apr. 12, 2021
(86) PCT No.: PCT/US2021/026824

§ 371 Date: Oct. 10, 2022

Related U.S. Application Data
(60) Provisional application No. 63/008,024, filed on Apr. 10, 2020.

(51) Int. Cl. A61M 25/02

(52) U.S. Cl. CPC A61M 25/02; A61M 2025/0246

(57) ABSTRACT

An apparatus (100), having: a first body (102); a second body (104); and a rotatable joint (108) that pivotally secures the first body to the second body, wherein in a closed configuration (200) the first body is folded onto the second body via the rotatable joint; a securing feature (110) that maintains the closed configuration; a catheter sheath feature (124) that is part of at least one of the first body and the second body that geometrically locks a catheter sheath (202) between the first body and the second body in the closed configuration; and a catheter feature (126) that is part of at least one of the first body and the second body and that frictionally locks a catheter (208) that is disposed in the catheter sheath between the first body and the second body in the closed configuration.

### FIELD OF THE INVENTION

The invention relates to a device that holds a catheter in position relative to a catheter sheath during a procedure.

### BACKGROUND OF THE INVENTION

Interventional radiologist and vascular surgeons perform procedures on patients including those who have acute blood clot formation in the vascular system. This disease entity is referred to as a thromboembolism. Often, a catheter designed to slowly infuse thrombolytic medication into the occluded vessel is positioned and left in place from 2 to 24 hours for the medication to adequately dissolve the blood clot. During this period of time, the patient is closely monitored in an acute care setting (ICU) for unexpected bleeding that may require the medication to be discontinued. The risk of life threatening bleeding is decreased by decreasing the dose of medication delivered, which is made possible by maintaining the exact positioning of the catheter throughout the treatment. The most common cause of catheter dislodgement is movement at the junction of the catheter and vascular sheath at the vascular access site of the patient. One known practice is to wrap tape around the catheter and then suture the tape to the sheath. This is often ineffective and increases the risk of needle stick injury to the physician. Accordingly, there is room for improvement in the art.

### BRIEF DESCRIPTION OF THE DRAWINGS

The invention is explained in the following description in view of the drawings that show:

FIG. 1 shows an example embodiment of the securing device disclosed herein and in a disassembled condition.

FIG. 2 shows the example embodiment of the securing device of FIG. 1 in an assembled condition and in an open configuration with a catheter sheath and a catheter disposed therein.

FIG. 3 shows the example embodiment of the securing device of FIG. 1 in an assembled condition and in a closed configuration with the catheter sheath and the catheter disposed therein.

FIG. 4 shows the securing device of FIG. 1 with example embodiments of anchoring devices.

### DETAILED DESCRIPTION OF THE INVENTION

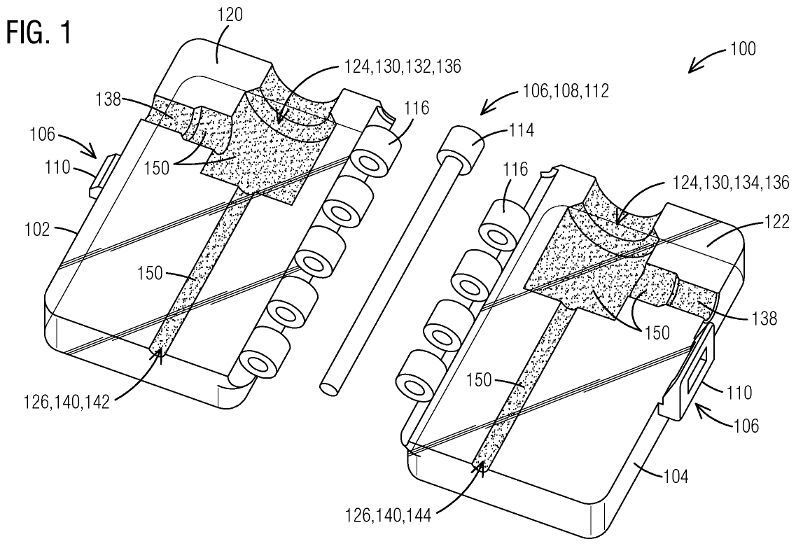

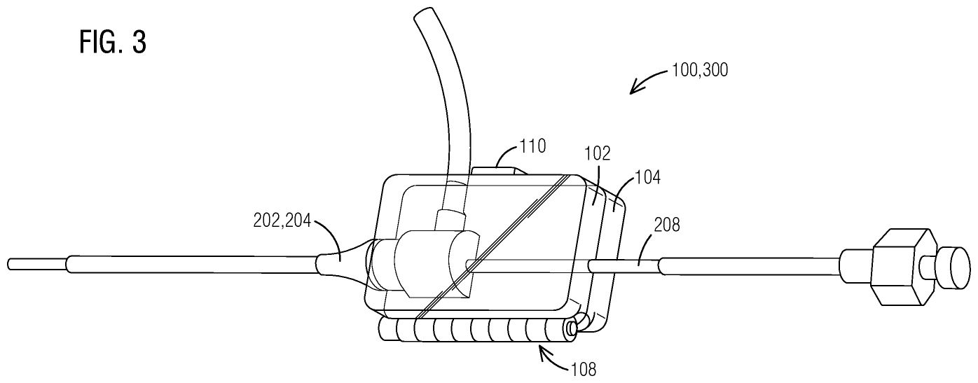

FIG. 1 shows a securing device 100 that includes a first body 102, a second body 104, and a securing arrangement 106. The securing arrangement may be any arrangement that is configured to secure the first body 102 to face the second body 104 when the securing device 100 is in a closed configuration (FIG. 3). In the example embodiment shown, the securing arrangement 106 includes a rotatable joint 108 that pivotally folds the first body 102 onto the second body 104 into the closed configuration (FIG. 3), and a securing feature 110 that maintains the closed configuration. In this example embodiment, the rotatable joint 108 includes a hinge 112 with a hinge pin 114 and knuckles 116. In this example embodiment, the securing feature 110 includes a snap having interlocking elements. The artisan will recognize that any suitable mechanism may be used as the securing feature, including physically interlocking configurations as well as magnetic etc. The folding action disclosed in this example embodiment to reach the closed configuration is, however, not necessary. The securing arrangement 106 is, at a minimum, configured to secure the first body 102 to face the second body 104 in the closed configuration. For example, the first body 102 may be disconnected from the second body 104 when not in the closed configuration. Hence, the rotatable joint is optional. The artisan will appreciate that there are other securing arrangements 106 suitable for holding the first body 102 to face the second body 104 and those are intended to be within the scope of the disclosure. For example, the rotatable joint could be replaced with a second snap. In an alternate example, the entire securing feature 110 could be, for example, a band or clamp that holds the first body 102 facing a discrete second body 104 etc.

The first body 102 and the second body 104 can be made of any suitable material known to the artisan. For example, the first body 102 and the second body 104 can be made of a plastic. In an example embodiment, the material of the first body 102 and/or the second body 104 is transparent. This may aid in being able to observe the catheter sheath and catheter during a procedure. Being able to observe the catheter sheath and catheter makes it possible to detect, for example, unexpected bleeding.

The first body 102 includes a first body cooperating surface 120. The second body 104 includes a second body cooperating surface 122. The securing device 100 further includes a catheter sheath feature 124 and a catheter feature 126. The catheter sheath feature 124 is configured to geometrically lock the catheter sheath between the first body 102 and the second body 104 in the closed configuration. As show in FIG. 1, the catheter sheath feature 124 is a catheter sheath compartment 130 that is distributed between the first body 102 and the second body 104. The catheter sheath compartment 130 in this example embodiment is composed of a first body catheter sheath recess 132 recessed into in the first body cooperating surface 120 plus a second body catheter sheath recess 134 recessed into the second body cooperating surface 122. In this example embodiment, the entire catheter sheath compartment 130 has a shape that is a negative of a shape of the catheter sheath. This enables a form fit of the catheter sheath within the catheter sheath compartment 130 without collapsing the catheter sheath. In this embodiment, the shape of the catheter sheath includes a shape 136 of a hub of the catheter sheath plus a shape 138 of a side port of the catheter sheath. While there may be friction between the catheter sheath and the first body cooperating surface 120 and/or the second body cooperating surface 122, this friction is wholly unnecessary to retain the catheter sheath in the catheter sheath compartment 130. The geometric interlocking between the catheter sheath and the catheter sheath compartment 130 is the mechanism that traps the catheter sheath in the catheter sheath compartment 130 between the first body 102 and the second body 104.

While the catheter sheath compartment 130 is shown as being distributed between the first body 102 and the second body 104, it is also envisioned that the catheter sheath compartment 130 may be disposed solely in the first body 102, and hence formed solely by the first body cooperating surface 120. Likewise, it is envisioned that the catheter sheath compartment 130 may be disposed solely in the second body 104, and hence formed solely by the second body cooperating surface 122. In these latter two embodiments, the catheter sheath compartment 130 may have shape in which only a portion is a negative of the catheter sheath. This would be necessary to allow the catheter sheath to be lowered into the catheter sheath compartment 130. Moreover, it is possible that the catheter sheath compartment 130 has a shape that is entirely unrelated to the shape of the catheter sheath, but which nonetheless creates a geometric interlock with the catheter sheath and thereby traps the catheter sheath between the first body 102 and the second body 104.

The catheter feature 126 shown in this example embodiment is configured such that the first body 102 and/or the second body 104 directly contact the catheter and lock the catheter in position relative to the catheter sheath via friction between the first body cooperating surface 120 and/or the second body cooperating surface 122 in the closed configuration. However, it is also envisioned that a material 150 may be disposed between the first body 102 and/or the second body 104 and the catheter. In an embodiment, the material 150 may be disposed in some or all of the catheter sheath feature 124, in some or all of the catheter feature 126, or in some or all of both. The material 150 may enhance friction between the device and the catheter sheath and the catheter. In an example embodiment, the material 150 is a resilient material (e.g. silicone). The material 150 may also be clear.

As shown in FIG. 1, the catheter feature 126 is a catheter compartment 140 that is distributed between the first body 102 and the second body 104. The catheter compartment 140 in this example embodiment is composed of a first body catheter recess 142 recessed into in the first body cooperating surface 120 plus a second body catheter recess 144 recessed into the second body cooperating surface 122.

In this example embodiment, the entire catheter compartment 140 has a shape that is a negative of a shape of the catheter. This enables a form fit of the catheter within the catheter compartment 140 without collapsing/occluding the catheter. Since the catheter is an elongated element of relatively uniform and smooth shape, and since the catheter may be repositioned, a friction fit is preferred.

An amount of friction between the catheter compartment 140 and the catheter can be selected. In one embodiment, the amount of friction is selected to be relatively high so that the catheter cannot be moved manually by a technician when the securing device 100 is closed. In such an example embodiment, a portion of the catheter outside/upstream of the securing device 100 would buckle if a technician tried to push the catheter further into the patient. In another embodiment, the amount of friction is selected to be relatively lower so that the catheter remains in place during conditions encountered during normal treatment, but which can be moved/repositioned manually by a technician even when the securing device 100 is closed. In such an example embodiment, a portion of the catheter outside/upstream of the securing device 100 would not buckle if a technician tried to push the catheter further into the patient.

While the catheter compartment 140 is shown as being distributed between the first body 102 and the second body 104, it is also envisioned that the catheter compartment 140 may be disposed solely in the first body 102, and hence formed solely by the first body cooperating surface 120. Likewise, it is envisioned that the catheter compartment 140 may be disposed solely in the second body 104, and hence formed solely by the second body cooperating surface 122. In these latter two embodiment, the catheter sheath compartment 130 may have shape in which only a portion is a negative of the catheter. This would be necessary to allow the catheter to be lowered into the catheter sheath compartment 130. Moreover, it is possible that the catheter compartment 140 has a shape that is entirely unrelated to the shape of the catheter, but which nonetheless creates a frictional fit with the sheath and thereby traps the catheter between the first body 102 and the second body 104. It is also envisioned that material between the first body 102 and the second body 104 could have a shape that is a negative of the shape of the catheter. In such an embodiment, the first body 102 and the second body 104 may or may not also have a shape that is a negative of the shape of the catheter. In other words, the catheter compartment 140 may be disposed in any or all of the first body 102, the second body 104, and the material between the first body 102 and the second body 104 and the catheter.

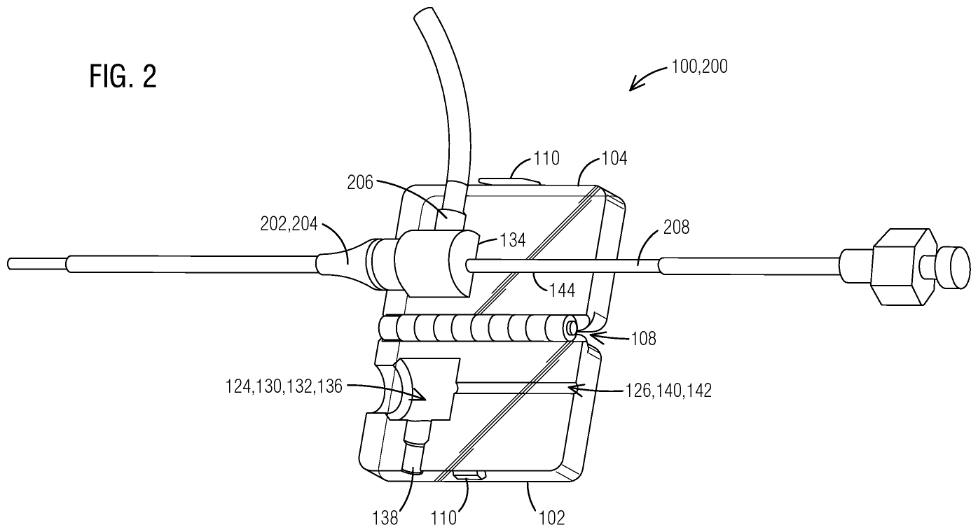

FIG. 2 shows the example embodiment of the securing device 100 of FIG. 1 in an assembled condition and in an open configuration 200 and disposed in which is a catheter sheath 202 having a hub 204 and a side port 206, and a catheter 208 disposed in the catheter sheath 202. The hub 204 of the catheter sheath 202 is shown disposed in the second body catheter sheath recess 134 and the catheter 208 is shown disposed in the second body catheter recess 144. A position of the catheter 208 relative to the catheter sheath 202 can be set before and/or after the catheter sheath 202 and the catheter 208 are positioned as shown. To secure the catheter 208 in a fixed position relative to the catheter sheath 202, the first body 102 is folded onto the second body 104 until the securing feature 110 engages and thereby holds the first body 102 in a fixed position relative to the second body 104, which traps the catheter sheath 202 and the catheter 208 therebetween.

FIG. 3 shows the example embodiment of the securing device 100 of FIG. 1 in an assembled condition and in a closed configuration 300 with the catheter sheath 202 and the catheter 208 disposed therein. In the closed configuration 300, the catheter sheath 202 is geometrically interlocked in the catheter sheath feature 124 and the catheter 208 is frictionally locked in position relative to the catheter sheath 202 by the catheter feature 126. In embodiments where the first body 102 and/or the second body 104 are made of transparent material like that shown, the transparent material enables one to see the catheter sheath 202 and the catheter 208 and thereby monitor conditions associated therewith.

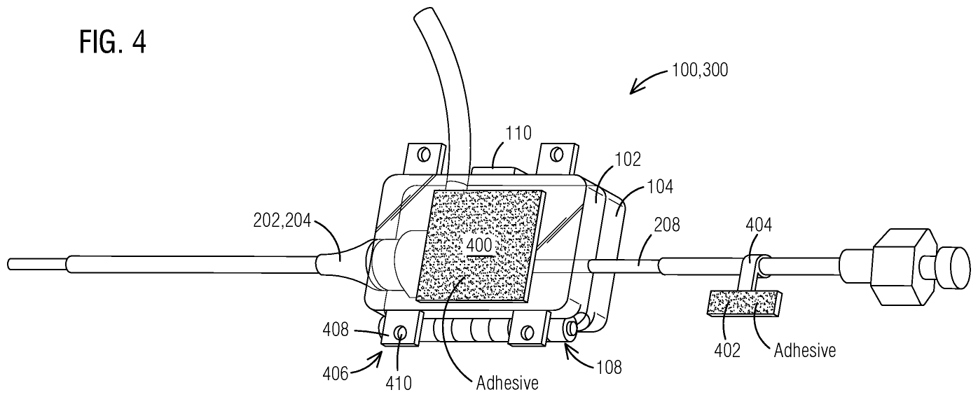

FIG. 4 shows the securing device 100 of FIG. 1 with example embodiments of anchoring devices. In an example embodiment, an anchoring device 400 includes adhesive that is secured to the first body 102, the second body 104, or both (which allows for a choice of which side to face the patient). The anchoring device 400 can also be adhesively secured to a patient so that the securing device 100 does not move relative to an insertion site on the patient. The anchoring device 400 can be as simple as a layer of adhesive that adheres directly to the first body 102, the second body 104, or both (which allows for choice of which side to face the patient) and directly to the patient. In such a configuration, there may or may not be an elevated platform on the apparatus 100 intended to receive the adhesive. The anchoring device 400 can be a double-sided adhesive (substrate with adhesive material on opposite sides). The double-sided adhesive may include a removable cover on a side to be adhered to the patient that can be removed immediately prior to being adhered to the patient. The anchoring device 400 may also include a combination of an adhesive and a mechanical connection. In such an example embodiment, the anchoring device 400 is adhesively secured to the first body 102, the second body 104, or both and mechanically secured to the patient via, for example, straps (elastic or inelastic) or the like.

In an alternate example embodiment, an anchoring device 402 includes an adhesive that is secured to the patient and includes a mechanical connection 404 configured to be secured to the first body 102, the second body 104, the catheter sheath 202, the hub 204, the side port 206, and/or the catheter 208 so that the securing device 100 does not move relative to an insertion site on the patient.

Examples of suitable anchoring devices include: the Stayfit® Fixation device manufactured by Merit Medical of Jordan Utah; the Statlock® Foley Stabilization Device manufactured by Bard Medical of Covington Ga.; the Statlock® PICC Plus Stabilization Device manufactured by Bard Medical of Covington Ga.; and the Revolution™ catheter securement device manufactured by Merit Medical of Jordan Utah.

In an alternate example embodiment, there may be one or more anchoring devices 406, each including an anchor tab 408 secured to the first body 102, the second body 104, or both so that the securing device 100 does not move relative to an insertion site on the patient. The anchor tab 408 may be suture-permeable so that the anchor tab 408 can be sutured directly to a patient through any part of the anchor tab 408. Alternately, or in addition, the anchor tab 408 may include a through-opening 410 through which a suture may pass and anchor the anchor tab 408 to the patient. The securing device 100 may include any combination of the anchoring devices disclosed above.

From the foregoing, it can be seen that the Inventor has devised an effective device that can hold a catheter in position relative to a catheter sheath during a procedure, which represents an improvement in the art.

While various embodiments of the present invention have been shown and described herein, it will be obvious that such embodiments are provided by way of example only. Numerous variations, swapping of features among embodiments, changes, and substitutions may be made without departing from the invention herein. Accordingly, it is intended that the invention be limited only by the spirit and scope of the appended claims.

1. An apparatus, comprising:
a first body; a second body; and a rotatable joint that pivotally secures the first body to the second body; wherein in a closed configuration the first body is folded onto the second body via the rotatable joint;
a securing feature that maintains the closed configuration;
a catheter sheath feature that is part of at least one of the first body and the second body that geometrically locks a catheter sheath between the first body and the second body in the closed configuration; and
a catheter feature that is part of at least one of the first body and the second body and that frictionally locks a catheter that is disposed in the catheter sheath between the first body and the second body in the closed configuration, wherein the rotatable joint optionally comprises a hinge.

2. (canceled)

3. The apparatus of claim 1, wherein in the closed configuration a first body cooperating surface of the first body faces a second body cooperating surface of the second body, and wherein the catheter sheath feature comprises a catheter sheath compartment in at least one of the first body cooperating surface and the second body cooperating surface in which the catheter sheath is trapped in the closed configuration.

4. The apparatus of claim 1, wherein in the closed configuration a first body cooperating surface of the first body faces a second body cooperating surface of the second body, and wherein the catheter sheath feature comprises a catheter sheath compartment distributed between the first body cooperating surface and the second body cooperating surface in which the catheter sheath is trapped in the closed configuration, wherein the catheter sheath compartment optionally comprises a catheter sheath compartment shape that is a negative of a shape of the catheter sheath.

5. (canceled)

6. The apparatus of claim 4, wherein the shape of the catheter sheath comprises a shape of a hub of the catheter sheath and a shape of a side port of the catheter sheath.

7. The apparatus of claim 1, wherein in the closed configuration a first body cooperating surface of the first body faces a second body cooperating surface of the second body, and wherein the catheter feature comprises a catheter compartment in at least one of the first body cooperating surface and the second body cooperating surface in which the catheter is trapped in the closed configuration.

8. The apparatus of claim 1, wherein in the closed configuration a first body cooperating surface of the first body faces a second body cooperating surface of the second body, and wherein the catheter feature comprises a catheter compartment distributed between the first body cooperating surface and the second body cooperating surface in which the catheter is trapped in the closed configuration, wherein the catheter compartment optionally comprises a catheter compartment shape that is a negative of a shape of the catheter.

9. (canceled)

10. An apparatus, comprising:
a first body; a second body; and a securing arrangement configured to secure the first body to face the second body in a closed configuration;
a catheter sheath feature configured to geometrically interlock a catheter sheath with at least one of the first body and the second body in the closed configuration; and
a catheter feature configured to frictionally engage a catheter with at least one of the first body and the second body in the closed configuration when the catheter is disposed in the catheter sheath.

11. The apparatus of claim 10, wherein the securing arrangement comprises:
a rotatable joint that pivotally folds the first body onto the second body in the closed configuration; and
a securing feature that maintains the closed configuration.

12. The apparatus of claim 10, wherein the catheter sheath feature comprises a catheter sheath compartment in at least one of the first body and the second body.

13. The apparatus of claim 10, wherein the catheter sheath feature comprises a catheter sheath compartment distributed between both the first body and the second body.

14. The apparatus of claim 13, wherein the catheter sheath feature comprises a catheter sheath shape that comprises a negative of a shape of the catheter sheath.

15. The apparatus of claim 10, wherein the catheter feature comprises a catheter compartment in at least one of the first body and the second body.

16. The apparatus if claim 10, wherein the catheter feature comprises a catheter compartment distributed between both the first body and the second body, wherein the catheter feature comprises a catheter feature shape that comprises a negative of a shape of the catheter, or wherein the catheter sheath feature optionally comprises a catheter sheath shape that comprises a negative of a shape of the catheter sheath.

17. (canceled)

18. (canceled)

19. (canceled)

20. The apparatus of claim 10, further comprising an anchoring feature configured to anchor the apparatus in place on a patient, wherein the anchoring feature optionally comprises an adhesive configured to adhere the apparatus to the patient.

21. (canceled)

22. The apparatus of claim 11, wherein the anchoring feature comprises an anchor tab configured to receive a suture that sutures the anchoring feature to the patient.

23. An apparatus, comprising:
a first body comprising a first body cooperating surface; and a second body comprising a second body cooperating surface that faces the first body cooperating surface when the apparatus is in a closed configuration; and
a securing arrangement configured to secure the first body to face the second body around a catheter sheath and around a catheter disposed in the catheter sheath in the closed configuration;
a catheter sheath feature disposed in at least the first body cooperating surface that traps the catheter sheath between the first body and the second body via a geometric interference; and
a catheter feature disposed in at least the first body cooperating surface, wherein in the closed configuration the catheter feature holds the catheter in position via friction but does not occlude the catheter.

24. The apparatus of claim 23, wherein the catheter sheath feature is distributed between the first body cooperating surface and the second body cooperating surface.

25. The apparatus of claim 23, wherein the catheter feature is distributed between the first body cooperating surface and the second body cooperating surface.

26. The apparatus of claim 23, wherein at least one of the first body and the second body comprise a material that is transparent, thereby enabling a user to see at least one of the catheter sheath and the catheter in the closed configuration.
